(19) United States
(12) Patent Application Publication
(10) Pub. No.: US 2021/0186883 A1
(43) Pub. Date: Jun. 24, 2021

(54) NOVEL PENTOSAN POLYSULFATE SODIUM PREPARATION

(71) Applicant: ReqMed Company, LTD., Tokyo (JP)

(72) Inventor: Tadashi MATSUMOTO, Tokyo (JP)

(21) Appl. No.: 17/269,860

(22) PCT Filed: Aug. 20, 2018
(86) PCT No.: PCT/JP2018/030666

§ 371 Date: Feb. 19, 2021

(51) Int. Cl. A61K 9/19; A61K 31/737; A61K 9/00

(52) U.S. Cl. CPC A61K 9/19; A61K 9/0019; A61K 31/737

(57) ABSTRACT

While developing a therapeutic agent for human arthritis by using a pentosan polysulfate sodium injection, the inventors of the present invention found that minute particles were generated due to delamination in an ampule of a pentosan polysulfate sodium injection for treatment of human arthritis. Although a pentosan polysulfate sodium injection has been used over the past 30 or more years, such findings had not been obtained. Accordingly, the present invention was achieved in order to prevent the occurrence of delamination in a pentosan polysulfate sodium injection. Based on the thought that using a lyophilized pentosan polysulfate sodium preparation makes it possible to prevent delamination in a pentosan polysulfate sodium injection, the inventors of the present invention investigated various lyophilized preparations and various methods for manufacturing the lyophilized preparations. As a result, the inventors of the present invention succeeded in producing a lyophilized pentosan polysulfate sodium preparation for the first time, and thus the present invention was completed.

### TECHNICAL FIELD

The present invention relates to a lyophilized pentosan polysulfate preparation.

### BACKGROUND ART

Pentosan polysulfate sodium is a semisynthetic compound obtained by chemically modifying polysaccharides extracted from European beeches, and was initially developed as an anticoagulant in Germany. Currently, a liquid injection (SP54 (registered trademark)) and an encapsulated oral preparation (Elmiron (registered trademark)) are marketed as therapeutic agents for interstitial cystitis. Moreover, pentosan polysulfate sodium has been known to have an effect of treating arthritis in animals (Non-Patent Document 1). A lyophilized pentosan polysulfate preparation has not been known in the past.

### CITATION LIST

### Non-Patent Document

Non-Patent Document 1: Wijekoon et al., BMC veterinary Research (2018) 14: 152

### SUMMARY OF INVENTION

### Technical Problem

While developing a therapeutic agent for human arthritis by using a pentosan polysulfate injection, the inventors of the present invention found that minute particles were generated due to delamination in an ampule of a pentosan polysulfate injection for treatment of human arthritis. Although a pentosan polysulfate injection has been used over the past 30 or more years, such findings had not been obtained. Accordingly, the present invention was achieved in order to prevent the occurrence of delamination in a pentosan polysulfate injection.

### Solution to Problem

Based on the thought that using a lyophilized pentosan polysulfate sodium preparation makes it possible to prevent delamination in a pentosan polysulfate injection, the inventors of the present invention investigated various lyophilized preparations and various methods for manufacturing the lyophilized preparations. As a result, the inventors of the present invention succeeded in producing a lyophilized pentosan polysulfate preparation for the first time and found that delamination did not occur when the lyophilized preparation was reconstituted, and thus the present invention was completed.

### DESCRIPTION OF EMBODIMENTS

In an aspect, the present invention relates to a lyophilized preparation containing pentosan polysulfate. In this specification, “pentosan polysulfate” is a polysaccharide that includes 1-4 linked β-D-xylanopyranose units as a basic skeleton and has a weight average molecular weight of 1000 to 6000 daltons or 1500 to 6000 daltons. For example, the pentosan polysulfate is represented by the formula below.

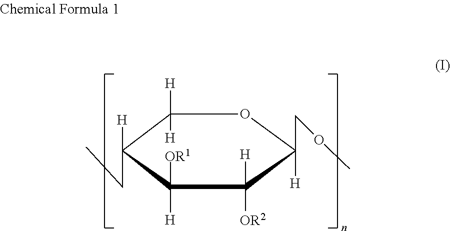

(In this formula, R¹ and R² represent SO₃H.)

It is known that, in the pentosan polysulfate, one R² per n of about 3 to 15 may be a 1-4 linked acetyl-substituted β-D-xylanopyranose group represented by the formula below. Accordingly, the pentosan polysulfate sodium in this specification may include a polysaccharide represented by the formula above in which one R² per n of 3 to 15 (preferably 6 to 12 or 8 to 10) on average is a 1-4 linked acetyl-substituted β-D-xylanopyranose group represented by the formula below.

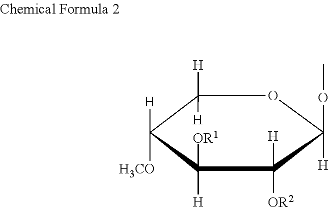

Pentosan polysulfate is described in Merck Index 11th Edition, Merck & Co, Inc., Rahway, N.J. (1989), p. 7093; U.S. Pat. Nos. 5,180,715 and 5,643,892; and U.S. Publication No. 2001/0034328.

The pentosan polysulfate of the present invention includes sulfate groups and can thus form a salt with a base. Accordingly, the pentosan polysulfate of the present invention may be a pharmacologically acceptable salt of pentosan polysulfate. The “pharmacologically acceptable salt” refers to a salt that is formed by the pentosan polysulfate of the present invention binding to an inorganic or organic base and is acceptable as a medicine to be administered to the body. Such salts are, for example, described in Berge et al., J. Pharm. Sci. 66: 1-19 (1977) and so on. Examples of the salts include salts formed with alkali metals and alkali earth metals such as zinc, lithium, sodium, potassium, magnesium, calcium, silver, lead, copper, gold, palladium, and barium; salts formed with amines such as ammonia, methylamine, dimethylamine, trimethylamine, dicyclohexylamine, tris(hydroxymethyl)aminomethane, N,N-bis(hydroxyethyl)piperazine, 2-amino-2-methyl-1-propanol, ethanolamine, N-methylglucamine, and L-glucamine; and salts with basic amino acids such as lysine, 6-hydroxylysine, and arginine. Specific examples thereof include pentosan polysulfate sodium, pentosan polysulfate calcium, and pentosan polysulfate potassium, and pentosan polysulfate sodium is preferable.

There is no particular limitation on the content of pentosan polysulfate sodium in the lyophilized preparation of the present invention as long as desired safety and effects can be achieved in an aqueous preparation after reconstitution. For example, the lyophilized preparation of the present invention contains pentosan polysulfate sodium (R¹ and R² represent SO₃Na in Formula (I)) such that its concentration after reconstitution is 80 to 120 mg/mL, and preferably 90 to 110 mg/mL, 95 to 105 mg/mL, or 100 mg/mL.

It is preferable that the lyophilized preparation of the present invention contains a buffer. In this specification, the “buffer” means a substance that serves to adjust a pH to be within a target value range when the lyophilized preparation of the present invention is reconstituted and that can be administered as a medicine to mammals. Examples of the buffer include, but are not limited to, a borate buffer (containing sodium borate and sodium hydroxide), a phosphate buffer (containing sodium dihydrogen phosphate and disodium hydrogen phosphate), a carbonate-bicarbonate buffer (containing sodium carbonate and sodium hydrogen carbonate), a citrate buffer (containing sodium citrate and citric acid), an acetate buffer (containing acetic acid and sodium acetate), a succinate buffer, a histidine buffer, a tartrate buffer, HBSS, tris(hydroxymethyl)aminomethane (THAM), a citrate/phosphate buffer, a barbital buffer, a Britton-Robinson buffer, a cacodylate buffer, a collidine buffer, a formate buffer, a maleate buffer, a McIlvaine buffer, a Prideaux-Ward buffer, a citrate-phosphate-borate buffer (Teorell-Stanhagen buffer), a veronal acetate buffer, a MES (2-(N-morpholino)ethanesulfonic acid) buffer, a BIS-TRIS (bis(2-hydroxyethyl)iminotris(hydroxymethyl)methane) buffer, an ADA (N-(2-acetamido)-2-iminodiacetic acid) buffer, an ACES (N-(carbamoylmethyl)-2-aminoethanesulfonic acid (sulfonaic acid)) buffer, a PIPES (piperazine-N, N′-bis(2-ethanesulfonic acid)) buffer, a MOPSO (3-(N-morpholino)-2-hydroxypropanesulfonic acid) buffer, a BIS-TRIS PROPANE (1,3-bis(tris(hydroxymethyl)methylamino) propane) buffer, a BES (N,N-bis(2-hydroxyethyl)-2-aminoethanesulfonic acid (sulfonaic acid)) buffer, a MOPS (3-(N-morpholino)propanesulfonic acid) buffer, a TES (N-tris(hydroxymethyl)methyl-2-aminoethanesulfonic acid) buffer, a HEPES (N-(2-hydroxyethyl)piperazine-N′-2-ethanesulfonic acid) buffer, a DIPSO (3-(N,N-bis(2-hydroxyethyl)amino)-2-hydroxypropanesulfonic acid) buffer, a MOBS (4-(N-morpholino)butanesufonic acid) buffer, a TAPSO (3-(N-tris(hydroxymethyl)methylamino)-2-hydroxypropanesufonic acid) buffer, a tris(hydroxymethylaminomethane) buffer, a HEPPSO (N-(2-hydroxyethyl)piperazine-N′-2-hydroxypropanesufonic acid) buffer, a POPSO (piperazine-N,N′-bis(2-hydroxypropanesulfonic acid)) buffer, a TEA (triethanolamine) buffer, an EPPS (N-(2-hydroxyethyl)piperazine-N′-3-propanesulfonic acid) buffer, a TRICINE (N-tris(hydroxymethyl)methylglycine) buffer, a GLY-GLY (glycylglycine) buffer, a BICINE (N,N-bis(2-hydroxyethyl)glycine) buffer, a HEPBS (N-(2-hydroxyethyl)piperazine-N′-(4-butanesulfonic acid)) buffer, a TAPS (N-tris(hydroxymethyl)methyl-3-aminopropanesufonic acid) buffer, and an AMPD (2-amino-2-methyl-1,3-propanediol) buffer, and a phosphate buffer is preferable.

There is no particular limitation on the content of the buffer in the lyophilized preparation of the present invention as long as desired safety and effects can be achieved in an aqueous preparation after reconstitution. For example, the lyophilized preparation of the present invention contains a buffer such that the pH after reconstitution is 4.8 to 7.4, 5.0 to 7.2, 5.0 to 7.0, or 5.2 to 7.0.

For example, when the lyophilized preparation of the present invention contains a phosphate buffer, the lyophilized preparation can contain disodium hydrogen phosphate and sodium dihydrogen phosphate such that their concentrations after reconstitution are 0.43 to 1.3, 0.5 to 1.2, 0.6 to 1.1, 0.7 to 1.0, or 0.87 mg/mL and 2.6 to 7.9, 3 to 7, 4 to 6, 5.0 to 5.5, or 5.26 mg/mL, respectively.

The lyophilized preparation of this specification contains a pH adjuster as needed. Examples of the pH adjuster include potassium hydroxide, sodium hydroxide, lactic acid, hydrochloric acid, adipic acid, aqueous ammonia, dry sodium carbonate, diluted hydrochloric acid, a citric acid hydrate, a sodium citrate hydrate, sodium dihydrogen citrate, glycine, glucono-δ-lactone, gluconic acid, crystalline sodium dihydrogen phosphate, succinic acid, acetic acid, ammonium acetate, a sodium acetate hydrate, diisopropanolamine, tartaric acid, D-tartaric acid, L-sodium tartrate, calcium hydroxide, magnesium hydroxide, sodium hydrogen carbonate, a sodium carbonate hydrate, triisopropanolamine, triethanolamine, carbon dioxide, a calcium lactate hydrate, sodium lactate, glacial acetic acid, monosodium fumarate, sodium propionate, boric acid, ammonium borate, borax, maleic acid, anhydrous citric acid, anhydrous sodium acetate, anhydrous sodium monohydrogen phosphate, anhydrous sodium dihydrogen phosphate, meglumine, methanesulfonic acid, monoethanolamine, sulfuric acid, a potassium aluminum sulfate hydrate, DL-malic acid, phosphoric acid, trisodium phosphate, a sodium hydrogen phosphate hydrate, dipotassium phosphate, potassium dihydrogen phosphate, and sodium dihydrogen phosphate.

The lyophilized preparation of the present invention may further contain a cryoprotectant in order to maintain the stability, or may not contain such a cryoprotectant. In particular, common lyophilized preparations require a cryoprotectant such as a saccharide (e.g., maltose or mannitol) to maintain the stability, whereas the lyophilized preparation of this specification contains no cyroprotectants but can be provided as a stable preparation. Accordingly, the present invention includes a lyophilized preparation containing no cryoprotectants. When the lyophilized preparation of the present invention contains a cryoprotectant, examples thereof include saccharides (e.g., maltose and mannitol).

For example, the lyophilized preparation of the present invention contains pentosan polysulfate sodium, disodium hydrogen phosphate dodecahydrate, and sodium dihydrogen phosphate dihydrate such that their concentrations after reconstitution are 80 to 120 mg/mL (preferably 90 to 110 mg/mL, 95 to 105 mg/mL, or 100 mg/mL), 1 to 4 mg/mL, and 4.5 to 9.0 mg/mL, respectively. More preferably, the lyophilized preparation of the present invention contains pentosan polysulfate sodium, disodium hydrogen phosphate dodecahydrate, and sodium dihydrogen phosphate dihydrate such that their concentrations after reconstitution are 100 mg/mL, 2.2 mg/mL, and 6.84 mg/mL, respectively. Alternatively, the lyophilized preparation of the present invention may contain pentosan polysulfate sodium, disodium hydrogen phosphate dodecahydrate, and sodium dihydrogen phosphate dihydrate at a mass ratio of 800 to 1200:16 to 32:32 to 104, or at a mass ratio of 1000:22:68.4. Alternatively, the lyophilized preparation of the present invention may contain pentosan polysulfate sodium, disodium hydrogen phosphate, and sodium dihydrogen phosphate at a mass ratio of 800 to 1200:6 to 12:24 to 80, or at a mass ratio of 1000:8.72:52.6.

The lyophilized preparation of the present invention can be manufactured by lyophilizing an aqueous solution containing pentosan polysulfate sodium and additives such as a buffer.

Pentosan polysulfate sodium can be obtained by reacting xylan extracted from bark of a plant such as beech with a sulfating agent such as chlorosufonic acid or chlorosufuric acid to form a sulfate and treating the resultant sulfate using sodium hydroxide. Also, pentosan polysulfate sodium can be manufactured in consideration of U.S. Pat. Nos. 2,689,848, 5,668,116, and U.S. Publication No. 2009/0111771. Moreover, novel methods for manufacturing pentosan polysulfate sodium (WO2008/107906; WO2009/047699; WO2012/101544; WO2009/087581) are also reported, and pentosan polysulfate sodium may be manufactured in consideration of these methods.

For example, the lyophilized preparation of the present invention can be manufactured as a preparation that is stabler or is easy to reconstitute by lyophilizing an aqueous solution containing pentosan polysulfate sodium and a buffer at concentrations that are 0.25 to 0.75 times (preferably 0.5 times) as high as those after reconstitution. For example, the lyophilized preparation of the present invention can be manufactured by lyophilizing an aqueous solution containing pentosan polysulfate sodium at a concentration of 25 to 75 mg/mL (preferably 30 to 70, 40 to 60, or 50 mg/mL), disodium hydrogen phosphate dodecahydrate at a concentration of 0.55 to 1.65 mg/mL (preferably 0.6 to 1.6, 0.8 to 1.4, or 1.1 mg/mL), and sodium dihydrogen phosphate dihydrate at a concentration of 1.71 to 5.13 mg/mL (preferably 1.8 to 5.0, 2.0 to 4.0, or 3.42 mg/mL).

In an aspect, the present invention encompasses such an aqueous solution to be used to manufacture a lyophilized preparation. Specifically, the aqueous solution to be used to manufacture the lyophilized preparation of the present invention is an aqueous solution containing pentosan polysulfate sodium and a buffer at concentrations that are 0.25 to 0.75 times (preferably 0.5 times) as high as those after reconstitution. For example, the aqueous solution to be used to manufacture the lyophilized preparation of the present invention contains pentosan polysulfate sodium at a concentration of 25 to 75 mg/mL (preferably 30 to 70, 40 to 60, or 50 mg/mL), disodium hydrogen phosphate dodecahydrate at a concentration of 0.55 to 1.65 mg/mL (preferably 0.6 to 1.6, 0.8 to 1.4, or 1.1 mg/mL), and sodium dihydrogen phosphate dihydrate at a concentration of 1.71 to 5.13 mg/mL (preferably 1.8 to 5.0, 2.0 to 4.0, or 3.42 mg/mL). Alternatively, the aqueous solution to be used to manufacture the lyophilized preparation of the present invention contains pentosan polysulfate sodium at a concentration of 25 to 75 mg/mL (preferably 30 to 70, 40 to 60, or 50 mg/mL), disodium hydrogen phosphate at a concentration of 0.22 to 1.65 mg/mL (preferably 0.6 to 1.6, 0.8 to 1.4, or 1.1 mg/mL), and sodium dihydrogen phosphate dihydrate at a concentration of 1.71 to 5.13 mg/mL (preferably 1.8 to 5.0, 2.0 to 4.0 mg/mL, or 3.42 mg/mL).

Lyophilization can commonly include a preliminary freezing step, a primary drying step, and a secondary drying step. In the preliminary freezing step, freezing is commonly performed at a temperature that is lower than or equal to the eutectic point. For example, the preliminary freezing step can be performed at a constant temperature, but the temperature can also be changed as appropriate. In this specification, the preliminary freezing step can preferably include a step performed at −40 to −50° C. for 30 minutes to 3 hours, a step performed at −10 to −20° C. for 1 to 10 hours, and a step performed at −40 to −50° C. for 30 minutes to 3 hours in the stated order.

The primary drying step is a step of sublimating frozen water under reduced pressure. It is known that water boils at 0° C. under a pressure of 610.6 Pa, and a sublimation temperature decreases as the atmospheric pressure decreases. In this specification, the primary drying step is preferably performed at 1 to 100 Pa and −10° C. to −20° C., more preferably at 2 to 50 Pa and −12° C. to −18° C., and most preferably at 10 Pa and −14° C. There is no particular limitation on the period of the primary drying step as long as all of the contained water can be sublimated, and the period can be set to 35 to 50 hours or 40 to 45 hours, for example. This may also be applied to a case of ten thousand 2.5-mL ampules.

The secondary drying step is a dehumidifying step for removing remaining water by increasing the temperature at lower pressure. In this specification, the secondary drying step is preferably performed by gradually increasing the temperature under a full vacuum environment. For example, the secondary drying step may be performed by increasing the temperature to 35 to 40° C. under a full vacuum environment and then keeping the temperature at 35 to 40° C. for 12 hours.

The sterilization degree or stability of the obtained lyophilized preparation can be maintained through capping, aluminum cap sealing, or the like, as needed.

This lyophilized preparation is excellent in stability required to supply the lyophilized preparation as a medicine. Specifically, the lyophilized preparation of this specification is stable at 40±2° C. and 75% RH, which are known as the conditions of an acceleration test, for at least 6 months. In the medicine stability test, one month in an acceleration test is considered to correspond to 6 months of storage at ordinary temperatures and pressures, and therefore, this lyophilized preparation is stable at ordinary temperatures and pressures for 36 months. It is possible to confirm whether or not a lyophilized preparation to be tested is stable as described above by storing the lyophilized preparation at 40±2° C. and 75% RH for 6 months and then analyzing whether or not the lyophilized preparation itself and a solution obtained by reconstituting the lyophilized preparation satisfy the specifications set for the preparation. When the preparation satisfies the specifications after the storage, it is determined that the preparation is stable under such storage conditions. Examples of the items of such specifications include the extemal appearance, the clarity and color, the average mass, the uniformity of mass, the uniformity of an administration unit, the mixing of minute particles (into the reconstituted solution): invisible minute particles, the mixing of minute particles (into the reconstituted solution): visible minute particles, the pH value (of the reconstituted solution), the drying loss, the transparency, the identification of a phosphate, the identification of pentosan polysulfate sodium (PPS) by gel permeation chromatography (GPC) and wet chemical analysis, the purity of sodium sulfate (IC), the purity (GPC), the purity of calcium, the PPS assay (GPC), the sterility, and the bacterial endotoxin. The following describes specific examples of the standards of these items: the external appearance: a white to bright yellow lyophilized product; the clarity and color colorless to light yellow clear solution; the uniformity of mass: up to ±10% for 18 ampules, and up to ±20% for 2 ampules (average mass deviation); the acceptable value (AV) of the uniformity of an administration unit (Ph. Eur. 2.9.40): based on Ph. Eur.; the mixing of minute particles (into the reconstituted solution) (invisible minute particles) (Ph. Eur. 2.9.19): up to 6000 minute particles with a diameter of 10 μm or more per ampule, and up to 600 minute particles with a diameter of 25 μm or more per ampule; the mixing of minute particles (into the reconstituted solution) (visible minute particles) (Ph. Eur. 2.9.20): no minute particles contained; the pH value (of the reconstituted solution) (Ph. Eur. 2.2.3): 5.2 to 7.0; the identification of a phosphate (Ph. Eur. 2.3.1): Yes; the identification of PPS (GPC): retention time of a main peak in a chromatogram of a sample solution corresponds to retention time of a standard solution; the identification of PPS (wet chemical analysis): red to purple; the purity of sodium sulfate (IC): less than 3% when calculated from the applied PPS content; the purity (GPC): no additional peaks; the PPS assay (GPC): 95.0 to 105.0% when calculated from the applied PPS content; the sterility (Ph. Eur. 2.6.1): based on Ph. Eur.: and the bacterial endotoxin (Ph. Eur. 2.6.14): less than 300 IU/ml.

The lyophilized preparation of the present invention can be reconstituted by adding a sterile aqueous diluent, preferably sterile water, thereto and mixing them. The present invention includes a method for preparing a liquid pharmaceutical composition containing pentosan polysulfate sodium, the method including reconstituting the above-mentioned lyophilized preparation in a sterile aqueous diluent. For example, the method for preparing a liquid pharmaceutical composition containing pentosan polysulfate sodium of the present invention includes adding a sterile aqueous diluent to a lyophilized preparation such that the concentration of pentosan polysulfate sodium is 80 to 120 mg/mL, and preferably 90 to 110 mg/mL, 95 to 105 mg/mL, or 100 mg/mL.

Also, the present invention encompasses a liquid pharmaceutical composition obtained through the reconstitution. In this specification, the terms “reconstituted solution”, “reconstituted liquid pharmaceutical composition”, and “liquid pharmaceutical composition obtained through reconstitution” are the same in meaning. It is preferable that the liquid pharmaceutical composition contains pentosan polysulfate sodium at a concentration of 80 to 120 mg/mL (preferably 90 to 110 mg/mL, 95 to 105 mg/mL, or 100 mg/mL), disodium hydrogen phosphate dodecahydrate at a concentration of 1.6 to 3.2 mg/mL, and sodium dihydrogen phosphate dihydrate at a concentration of 3.2 to 10.4 mg/mL. It is more preferable that the liquid pharmaceutical composition contains pentosan polysulfate sodium, disodium hydrogen phosphate dodecahydrate, and sodium dihydrogen phosphate dihydrate such that their concentrations after reconstitution are 100 mg/mL, 2.2 mg/mL, and 6.84 mg/mL, respectively. Also, the pH of the liquid pharmaceutical composition is preferably 5.0 to 7.0.

The lyophilized preparation or reconstituted liquid pharmaceutical composition of the present invention can be used for humans and mammals such as oxen, deer, horses, donkeys, wild boars, pigs, sheep, goats, dogs, cats, raccoon dogs, foxes, rabbits, mice, and squirrels for medical purposes (treatment purposes or prevention purposes). For example, the lyophilized preparation of the present invention can be applied to anticoagulants, or therapeutic agents or preventive agents for interstitial cystitis, osteoarthritis, lysosomal disease, and human lymphotropic virus type 1 (HTLV-1) associated myelopathy (HAM). For example, U.S. Pat. No. 9,155,784 discloses that pentosan polysulfate sodium has anti-TNFα activity and is effective at treating lysosomal disease. Lysosomal disease refers to a disease or disorder caused by the accumulation or presence of lysosomal enzymes due to abnormality in lysosomal enzymes. Examples of lysosomal disease include type-Il glycogenosis, Pompe disease, α-glucosidase (acid maltase) deficiency, sphingolipidosis, GM1 gangliosidosis, GM2 gangliosidosis (Tay-Sachs disease, Sandhoff disease), metachromatic leukodystrophy (MLD), Fabry disease, Farber disease, Gaucher disease, Niemann-Pick disease (A, B, C types), Krabbe disease, mucopolysaccharidosis, mucolipidosis, multiple sulfatase deficiency (MSD), sialidosis, galactosialidosis, I-cell disease, α-mannosidosis, β-mannosidosis, fucosidosis, aspartylglucosaminuria, Schindler disease, Wolman disease, Danon disease, free sialic acid storage disease, and ceroid lipofuscinosis.

In an aspect, the present invention includes a method for treating or preventing interstitial cystitis, osteoarthritis, lysosomal disease, or HAM, the method including administering an effective amount of a liquid pharmaceutical composition obtained by reconstituting the lyophilized preparation of the present invention to patients in need thereof. The term “patients in need thereof” means patients who are affected with or are likely to be affected with these diseases. Patients who are affected with these diseases and need to be treated are preferable. For example, when the above-mentioned liquid pharmaceutical composition is used for therapeutic purposes or preventive purposes, the liquid pharmaceutical composition can be administered in the oral administration form or in the parenteral administration form such as an injection or infusion. The dose varies depending on the symptom, age, sex, weight, administration form, and the like, and when the composition is orally administered, for example, the daily dose per adult is commonly 100 to 1000 mg.

Hereinafter, the present invention will be described more specifically based on examples. However, the present invention is not limited to these examples. It should be noted that all documents cited throughout the present application are hereby wholly incorporated herein by reference.

### EXAMPLES

### Example 1: Manufacturing of Lyophilized Pentosan Polysulfate Sodium Preparation

First, 33.22 g of disodium hydrogen phosphate dodecahydrate was added to 13.00 kg of water for injection, and then 103.28 g of sodium dihydrogen phosphate dihydrate was added thereto. Next, 1574.6 g of pentosan polysulfate sodium was added to the resultant phosphate buffer and dissolved. Water for injection was added thereto, and then the pH was adjusted to 5.9 to 6.1 using 1 mol/L hydrochloric acid and 1 mol/L sodium hydroxide. Water for injection was added such that the total quantity was 31.05 kg and the resultant solution was stirred. The solution was sterilized using a 0.22-μm filter and was then filled into vials such that each vial contained 2.570 g of the solution.

The vials were half-capped with rubber stoppers, and then lyophilization was performed using a K1 lyophilizer (DFB-60R-07ASC) under the following conditions.

Preliminary freezing: reducing the temperature from room temperature to −42° C. or lower (about −45° C.); keeping the temperature at −42° C. or lower (about −45° C.) for 1 hour; increasing the temperature to −14° C.; keeping the temperature at −14° C. for 3 hours; reducing the temperature from −14° C. to −42° C. or lower (about −45° C.); keeping the temperature at −42° C. (about −45° C.) for 1 hour

Primary drying: performed at a pressure of 10 Pa throughout, increasing the temperature from −42° C. or lower (about −45° C.) to −14° C. over 1.5 hours; keeping the temperature at −14° C. for 43 hours

Secondary drying: performed under reduced pressure (full vacuum condition) throughout, increasing the temperature from −14° C. to 37° C.; keeping the temperature at 37° C. for 12 hours

After recovery of the pressure through N2 substitution, the vials were taken out and sealed with aluminum caps. Thus, the lyophilized pentosan polysulfate sodium preparation was obtained.

### Example 2: Stability Test on Lyophilized Pentosan Polysulfate Sodium Preparation

The lyophilized pentosan polysulfate sodium preparation manufactured using the method of Example 1 was stored at 40° C. and 75% RH for 6 months, and then the stability thereof was evaluated (acceleration test).

Table 1 shows the results. It was shown that the lyophilized preparation met the standards of all of the evaluation items after 6-month storage. In particular, mixing of minute particles (visible minute particles) was never observed during the 6-month storage period, and thus it was also found that delamination did not occur.

TABLE 1

| | | | | | | Yes or |
|---|---|---|---|---|---|---|
| | | | | | | No: |
| Evaluation | | Initial | 1 month | 3 months | 6 months | meeting |
| items | Standards | analysis | after | after | after | standards |
| External | White to bright | White | White | White | White | Yes |
| appearance | yellow | lyophilized | lyophilized | lyophilized | lyophilized | |
| | lyophilized | product | product | product | product | |
| | product | | | | | |
| Clarity and | Colorless to | Light | Light | Light | Light | Yes |
| color | light yellow | yellow | yellow | yellow | yellow | |
| | clear solution | clear | clear | clear | clear | |
| | | solution | solution | solution | solution | |
| Average mass | Reference | 130.5 mg | 130.1 mg | 130.6 mg | 132.7 mg | Yes |
| Uniformity of | Up to ±10% | Yes | Yes | Yes | Yes | Yes |
| mass | for 18 | | | | | |
| | ampules, and | | | | | |
| | up to ±20% | | | | | |
| | for 2 ampules | | | | | |
| | (average | | | | | |
| | mass | | | | | |
| | deviation) | | | | | |
| Acceptable | Based on Ph. | Yes | Yes | Yes | Yes | Yes |
| value (AV) of | Eur. | | | | | |
| uniformity of | | | | | | |
| administration | | | | | | |
| unit | | | | | | |
| (Ph.Eur.2.9.40) | | | | | | |
| Mixing of | Up to 6000 | 35 minute | 97 minute | 56 minute | 35 minute | Yes |
| minute | minute | particles | particles | particles | particles | |
| particles (into | particles with | with | with | with | with | |
| reconstituted | diameter of 10 | diameter | diameter | diameter | diameter | |
| solution): | μm or more | of 10 μm | of 10 μm | of 10 μm | of 10 μm | |
| invisible | per ampule, | or more | or more | or more | or more | |
| minute | and up to 600 | per | per | per | per | |
| particles | minute | ampule, | ampule, | ampule; | ampule, | |
| (Ph.Eur.2.9.19) | particles with | and 4 | and | 37 and | 15 and 3 | |
| | diameter of 25 | minute | minute | minute | minute | |
| | μm or more | particles | particles | particles | particles | |
| | per ampule | with | with | with | with | |
| | | diameter | diameter | diameter | diameter | |
| | | of 25 μm | of 25 μm | of 25 μm | of 25 μm | |
| | | or more | or more | or more | or more | |
| | | per | per | per | per | |
| | | ampule | ampule | ampule | ampule | |
| Mixing of | No particles | No | No | No | No | Yes |
| minute | | particles | particles | particles | particles | |
| particles (into | | | | | | |
| reconstituted | | | | | | |
| solution.: | | | | | | |
| visible minute | | | | | | |
| particles | | | | | | |
| (Ph.Eur.2.9.20) | | | | | | |
| pH value (of | 5.2 to 7.0 | 5.9 | 5.9 | 5.9 | 6.0 | Yes |
| the | | | | | | |
| reconstituted | | | | | | |
| solution) | | | | | | |
| (Ph.Eur.2.2.3) | | | | | | |
| Drying loss | Reference | 2.5% | 16% | 1.8% | 2.2% | Yes |
| (Ph.Eur.2.2.32) | | | | | | |
| Transparency | Reference | 78.4% | 79.3% | 79.1% | 69.0% | Yes |
| Identification of | Yes | Yes | — | — | — | Yes |
| phosphate | | | | | | |
| (Ph.Eur2.3.1) | | | | | | |
| Identification of | Retention time | Yes | — | — | — | Yes |
| PPS (GPC) | of main peak | | | | | |
| | in | | | | | |
| | chromatogram | | | | | |
| | of sample | | | | | |
| | solution | | | | | |
| | corresponds | | | | | |
| | to retention | | | | | |
| | time of | | | | | |
| | standard | | | | | |
| | solution | | | | | |
| Identification of | Red to purple | Yes | — | — | — | Yes |
| PPS (wet | | | | | | |
| chemical | | | | | | |
| analysis) | | | | | | |
| Purity of | Less than 3% | 0.9% | 1.0% | 0.9% | 1.1% | Yes |
| sodium sulfate | when | | | | | |
| (IC) | calculated | | | | | |
| | from applied | | | | | |
| | PPS content | | | | | |
| Purity (GPC) | No additional | Yes | Yes | Yes | Yes | Yes |
| | peaks | | | | | |
| Purity of | Reference | 0.1% | — | — | — | Yes |
| calcium | | | | | | |
| PPS assay | 95.0 to | 101.3% | 101.0% | 100.0% | 99.0% | Yes |
| (GPC) | 105.0% when | | | | | |
| | calculated | | | | | |
| | from applied | | | | | |
| | PPS content | | | | | |
| Sterility | Based on Ph, | Yes | — | — | Yes | Yes |
| (Ph.Eur.2.6.1) | Eur. | | | | | |
| Bacterial | Less than 300 | Yes | — | — | Yes | Yes |
| endotoxin | IU/ml | | | | | |
| (ph.Eur.2.6.14) | | | | | | |

1. A lyophilized preparation comprising pentosan polysulfate or a salt thereof.

2. The lyophilized preparation according to claim 1, further comprising a buffer.

3. The lyophilized preparation according to claim 2, wherein the buffer is a phosphate buffer.

4. The lyophilized preparation according to any one of claims 1 to 3, comprising no cryoprotectants.

5. The lyophilized preparation according to any one of claims 1 to 4, which is stable at 40±2° C. and 75% RH for at least 6 months.

6. The lyophilized preparation according to any one of claims 1 to 5, wherein visible minute particles are not confirmed in a reconstituted liquid pharmaceutical composition of the lyophilized preparation.

7. The lyophilized preparation according to any one of claims 1 to 6, wherein the salt of pentosan polysulfate is pentosan polysulfate sodium.

8. The lyophilized preparation according to claim 7, wherein the concentration of the pentosan polysulfate sodium after reconstitution is 80 to 120 mg/mL.

9. The lyophilized preparation according to claim 7 or 8, comprising pentosan polysulfate sodium at a concentration of 80 to 120 mg/mL, disodium hydrogen phosphate dodecahydrate at a concentration of 1 to 4 mg/mL, and sodium dihydrogen phosphate dihydrate at a concentration of 4.5 to 9 mg/mL.

10. An aqueous solution for preparing a lyophilized pentosan polysulfate sodium preparation, the aqueous solution comprising pentosan polysulfate sodium at a concentration of 25 to 75 mg/mL.

11. The aqueous solution according to claim 10, comprising disodium hydrogen phosphate dodecahydrate at a concentration of 0.55 to 1.65 mg/mL, and sodium dihydrogen phosphate dihydrate at a concentration of 1.71 to 5.13 mg/mL.

12. A method for manufacturing the lyophilized preparation according to claim 7, comprising performing lyophilization on the aqueous solution according to claim 10 or

13. The manufacturing method according to claim 12, wherein the lyophilization includes a primary drying step performed at −10° C. to −20° C.

14. The manufacturing method according to claim 12 or 13,
wherein the lyophilization includes a preliminary freezing step including:
keeping a temperature at −40 to −50° C. for 30 minutes to 3 hours;
keeping the temperature at −10 to −20° C. for 1 to 10 hours; and
keeping the temperature at −40 to −50° C. for 30 minutes to 3 hours.

15. A lyophilized preparation obtained using the method according to any one of claims 12 to 14.

16. The lyophilized preparation according to any one of claims 1 to 9 and 15, which is a therapeutic agent or preventive agent for interstitial cystitis, osteoarthritis, lysosomal disease, or HAM, or an anticoagulant.

17. A liquid pharmaceutical composition obtained by reconstituting the lyophilized preparation according to any one of claims 1 to 9, 15, and 16.

18. The liquid pharmaceutical composition according to claim 17, comprising pentosan polysulfate sodium at a concentration of 100 mg/mL.

19. A method for preparing a liquid pharmaceutical composition comprising pentosan polysulfate or a salt thereof, the method comprising reconstituting the lyophilized preparation according to any one of claims 1 to 8, 15, and 16 in a sterile aqueous diluent.

20. A method for treating or preventing interstitial cystitis, osteoarthritis, lysosomal disease, or HAM, comprising administering an effective amount of a liquid pharmaceutical composition prepared using the preparing method according to claim 19 to a patient in need thereof.
